(19) United States
(12) Patent Application Publication
(10) Pub. No.: US 2021/0052414 A1
(43) Pub. Date: Feb. 25, 2021

(54) NASOPHARYNX STENT FOR ANTI-SNORING

(71) Applicant: XIAONIU HEALTH CO., LTD., Sanhe, Hebei (CN)

(72) Inventor: Hui YAN, Sanhe, Hebei (CN)

(21) Appl. No.: 17/044,197

(22) PCT Filed: Jun. 28, 2019
(86) PCT No.: PCT/CN2019/093745

§ 371 Date: Sep. 30, 2020

(30) Foreign Application Priority Data
Jun. 29, 2018 (CN) 201810712922.3
Jun. 29, 2018 (CN) 201821048453.1

(51) Int. Cl. A61F 5/56

(52) U.S. Cl. CPC A61F 5/56

(57) ABSTRACT

A nasopharynx stent (100, 200) for anti-snoring comprises a nasal tube (1) configured as a soft nasal tube; a balloon (2) configured as a soft balloon and communicated with a terminal end of the nasal tube (1). The balloon (2) is provided with a vent hole (23) in communication with the nasal tube (1) and first mesh-shaped through holes (21, 20) formed in a peripheral wall of the balloon (2).

### CROSS-REFERENCE TO RELATED APPLICATIONS

The present application is based on and claims priority to Chinese Patent Application Serial No. 201810712922.3 and No. 201821048453.1, filed on Jun. 29, 2018, the entire contents of which are incorporated herein by reference.

### FIELD

The present application relates to a technical field of medical instruments, and more particularly, to a nasopharynx stent for anti-snoring.

### BACKGROUND

OSAHS, short for obstructive sleep apnea-hypopnea syndrome, refers to apnea and hypoventilation caused by collapse and obstruction of the upper airway during sleep, accompanied by symptoms of snoring, disturbance of sleep structure, frequent decrease in blood oxygen saturation, daytime sleepiness, etc. OSAHS has a high incidence and great harm and is closely related to multiple systemic fatal diseases throughout the body. The fact that the upper airway has a narrow structure or is susceptible to collapse is the root cause of OSAHS. OSA (obstructive sleep apnea) accounts for up to 70% of people with OSAHS. There are four types according to the obstruction site:

Type I: the stenosis is above the nasopharynx (nasopharynx and nasal cavity);

Type II: the stenosis is in the oropharynx (soft palate and tonsil/uvula level);

Type III: the stenosis is in the hypopharynx (root of tongue and epiglottis level);

Type IV: the stenosis is in each of the above sites or in more than two sites.

The type II and type III account for the highest proportion. When your body is in a relaxed state during sleep, that part of tissue will sag, compressing your airways, narrowing them and causing breathing problems (such as snoring). Stopping breathing many times a night will cause various health problems.

For OSA patients, the current treatment methods are divided into surgical treatment and non-surgical treatment. The surgical treatment is complicated, highly risky, and easy to relapse. Therefore, the recognized first choice is a non-surgical therapy of continuous airway pressure (CPAP) with a respirator, which has a wide range of treatment types. However, the current respirators have some shortcomings, such as complicated structures, inconvenient use and high prices.

### SUMMARY

The present disclosure seeks to solve at least one of the technical problems existing in the related art. To this end, the present disclosure proposes a nasopharynx stent for anti-snoring effectively, which has a simple structure, convenient use, and low cost.

A nasopharynx stent for anti-snoring according to embodiments of the present disclosure includes: a nasal tube configured as a soft nasal tube; and a balloon configured as a soft balloon, communicated with a terminal end of the nasal tube, and provided with a vent hole in communication with the nasal tube and first mesh-shaped through holes formed in a peripheral wall of the balloon.

For the nasopharynx stent according to the embodiments of the present disclosure, when inserted into a nasopharyngeal passage of a human body until the balloon reaches a uvula (soft palate) of the human body, the balloon can withstand natural pressure at the uvula (soft palate) due to the appropriate softness of the soft balloon, and the balloon is sufficient to support collapse of the airway at this place, such that the airway is unobstructed by means of the vent hole in the balloon, which is conducive to air inhalation and exhalation and effectively stops snoring. The nasopharynx stent enables the inhaled air to fully contact with a mucous membrane of the cavity, generating a wetting effect, by means of the first mesh-shaped through holes in the balloon. In addition, the nasopharynx stent is simple in structure, convenient to use, and low in cost.

According to one embodiment of the present disclosure, the nasal tube is provided with a second mesh-shaped through hole.

According to one embodiment of the present disclosure, the nasopharynx stent further includes a baffle connected to a front end of the nasal tube.

Further, the baffle is provided with a plurality of ventilation holes.

Further, the nasal tube includes a first nasal tube and a second nasal tube; the balloon includes a first balloon and a second balloon; the first balloon is in communication with a terminal end of the first nasal tube while the second balloon is in communication with a terminal end of the second nasal tube; the baffle includes a first baffle and a second baffle; the first baffle is connected to a front end of the first nasal tube, while the second baffle is connected to a front end of the second nasal tube; the first baffle is connected with the second baffle.

According to one embodiment of the present disclosure, the nasopharynx stent further includes a nose pack having an adhesive backing, and the nose pack is connected with the baffle. By attaching the nose pack to the nose, the nasopharynx stent can be fixed.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and/or other aspects and advantages of embodiments of the present disclosure will become apparent and more readily appreciated from the following descriptions of embodiments made with reference to the drawings, in which:

FIG. 1 illustrates a schematic view of a nasopharynx stent for anti-snoring according to one embodiment of the present disclosure.

FIG. 2 illustrates a schematic view of a nasopharynx stent for anti-snoring according to another embodiment of the present disclosure.

FIG. 3 illustrates a schematic view of a baffle and a nose pack of a nasopharynx stent for anti-snoring according to another embodiment of the present disclosure.

FIG. 4 illustrates an application scenario of a nasopharynx stent for anti-snoring according to one embodiment of the present disclosure.

### DETAILED DESCRIPTION

Embodiments of the present disclosure will be described in detail below, and examples of the embodiments will be illustrated in the accompanying drawings. The same or similar reference numerals represent the same or similar elements or the elements having the same or similar functions throughout the descriptions. The embodiments described below with reference to the accompanying drawings are exemplary, are merely used to explain the present disclosure, and cannot be construed to limit the present disclosure.

In the description of the present disclosure, it should be understood that terms such as “central,” “longitudinal,” “transverse,” “length,” “width,” “thickness,” “up,” “down,” “front,” “rear,” “left,” “right,” “vertical,” “horizontal,” “top,” “bottom,” “inner,” “outer,” “clockwise,” “counterclockwise,” “axial,” “radial,” “circumferential” and the like should be construed to refer to the orientation as then described or as illustrated in the drawings under discussion. These terms are for convenience and simplification of description and do not indicate or imply that the device or element referred to must have a particular orientation, or be constructed and operated in a particular orientation, so these terms shall not be construed to limit the present disclosure. In addition, the feature defined with “first” and “second” may comprise one or more of this feature. In the description of the present disclosure, “a plurality of” means two or more than two, unless specified otherwise.

In the description of the present disclosure, it should be noted, unless specified or limited otherwise, the terms “mounted,” “connected,” “coupled” or the like are used broadly. The terms may be, for example, fixed connections, detachable connections, or integral connections; may also be mechanical or electrical connections, may also be direct connections or indirect connections via intervening structures; and may also be inner communications of two elements, which could be understood by those skilled in the art according to specific situations.

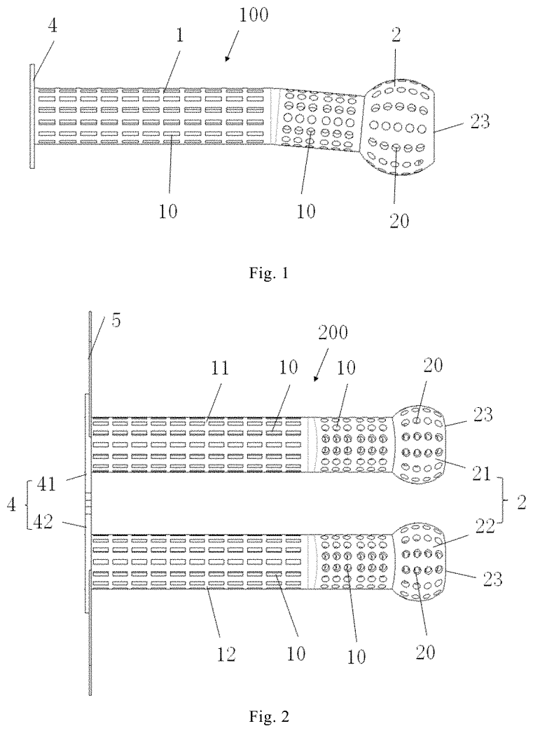

A nasopharynx stent for anti-snoring according to embodiments of the present disclosure will be described with reference to FIGS. 1-4.

As shown in FIG. 1, a nasopharynx stent 100 for anti-snoring according to an embodiment of the present disclosure includes: a nasal tube 1 configured as a soft nasal tube; and a balloon 2 configured as a soft balloon and communicated with a terminal end of the nasal tube 1. The balloon 2 is provided with a vent hole 23 in communication with the nasal tube 1 and first mesh-shaped through holes 21 formed in a peripheral wall of the balloon 2.

For the nasopharynx stent 100 according to the embodiment of the present disclosure, when inserted into a nasopharyngeal passage of a human body until the balloon 2 reaches a uvula (soft palate) of the human body (as shown in FIG. 4), the balloon 2 can withstand natural pressure at the uvula (soft palate), and the balloon 2 is sufficient to support collapse of the airway at this place, such that the airway is unobstructed by means of the vent hole 23 in the balloon 2, which is conducive to air inhalation and exhalation, and effectively stops snoring. Optionally, the nasopharynx stent 100 enables the inhaled air to fully contact with a mucous membrane of the cavity, generating a wetting effect, by means of the first mesh-shaped through holes 20 in the balloon 2. In addition, the nasopharynx stent 100 is simple in structure, convenient to use, and low in cost.

Optionally, the nasal tube 1 and the balloon 2 are made of medical-grade biocompatible soft materials, such that people can feel more comfortable to wear the stent.

According to one embodiment of the present disclosure, the nasal tube 1 is provided with second mesh-shaped through holes 10, such that the inhaled air can make full contact with an inner wall and the mucous membrane of the nasal cavity, which is conducive to wetting the mucous membrane of the nasal cavity and cleaning the air.

According to one embodiment of the present disclosure, the nasopharynx stent 10 further includes a baffle 4 connected to a front end of the nasal tube 1. Specifically, the baffle 4 is fitted over and connected to an outer tube wall of the front end of the nasal tube 1, and the baffle 4 is disposed along a radial plane of the nasal tube 1. Specifically, the baffle 4 may be a ring-shaped sheet, and an inner circumference of the baffle 4 fits with the outer wall of the nasal tube 1. The ring-shaped sheet can be embodied as a circle, an ellipse, or the like. The baffle 4 can prevent the nasopharynx stent from continuing to advance after the nasopharynx stent is inserted into the nasopharyngeal passage.

As shown in FIG. 2, a nasopharynx stent 200 for anti-snoring according to another embodiment of the present disclosure includes a nasal tube 1, a balloon 2, and a baffle 4. The nasal tube is a soft nasal tube and is provided with second mesh-shaped through holes 10. The balloon 2 is a soft balloon and is in communication with a terminal end of the nasal tube. The balloon 2 is provided with a vent hole 23 and first mesh-shaped through holes 20. The baffle 4 is fitted over and connected to an outer tube wall of a front end of the nasal tube 1. Specifically, the nasal tube includes a first nasal tube 11 and a second nasal tube 12; the balloon 2 includes a first balloon 21 and a second balloon 22; the first balloon 21 is in communication with a terminal end of the first nasal tube 11 while the second balloon 22 is in communication with a terminal end of the second nasal tube 12; the first balloon 21 and the second balloon 22 are both provided with the vent holes 23 and the first mesh-shaped through holes 20; the baffle 4 includes a first baffle 41 and a second baffle 42; the first baffle 41 is fitted over and connected to an outer tube wall of a front end of the first nasal tube 11, while the second baffle 42 is fitted over and connected to an outer tube wall of a front end of the second nasal tube 12; the first baffle 41 is connected with the second baffle 42. For the nasopharynx stent 200 according to another embodiment of the present disclosure, when the nasopharynx stent 200 is inserted into the nasopharyngeal passage of the human body until the balloon 2 reaches the uvula (soft palate) of the human body, the balloon can withstand natural pressure at the uvula (soft palate) due to the appropriate softness of the soft balloon 2, and the balloon 2 is sufficient to support collapse of the airway at this place, such that the airway is unobstructed by means of the vent hole 23 in the balloon 2, which is conducive to air inhalation and exhalation, and effectively stops snoring. The nasopharynx stent 200 enables the inhaled air to fully contact with the inner wall and the mucous membrane of the nasal cavity, bringing about wetting and cleaning effects, by means of the second mesh-shaped through holes 10 in the nasal tube and the first mesh-shaped through holes 20 in the balloon 2. In addition, the nasopharynx stent 100 is simple in structure, convenient to use, and low in cost. Optionally, the nasal tube and the balloon 2 are made of medical-grade biocompatible soft materials, such that people can feel more comfortable to wear the stent.

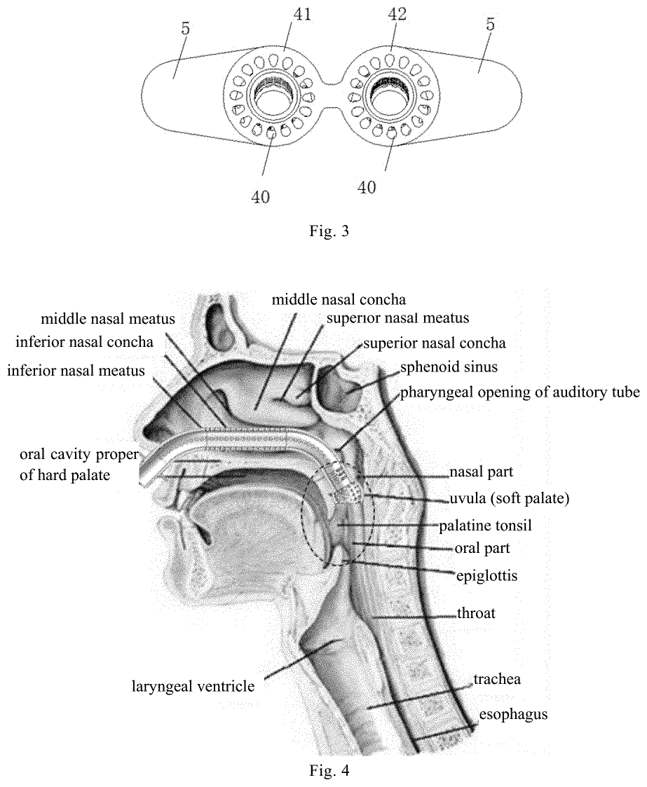

As shown in FIG. 3, according to still another embodiment of the present disclosure, the baffle 4 is provided with ventilation holes 40. Specifically, the first baffle 41 and the second baffle 42 are both provided with the ventilation holes 40.

As shown in FIG. 3, according to still another embodiment of the present disclosure, the nasopharynx stent 200 further includes a nose pack 5 having an adhesive backing, and the nose pack 5 is connected to the baffle 4. By attaching the nose pack 5 to the nose, the nasopharynx stent 200 can be fixed. Specifically, the nose pack 5 may be in a strip shape, a semi-ellipse shape, a “wing” shape, or the like.

Reference throughout this specification to “an embodiment,” “some embodiments,” “an exemplary embodiment,” “an example,” “a specific example,” or “some examples,” means that a particular feature, structure, material, or characteristic described in connection with the embodiment or example is included in at least one embodiment or example of the present disclosure. Thus, the appearances of the phrases in various places throughout this specification are not necessarily referring to the same embodiment or example of the present disclosure. Moreover, the particular features, structures, materials, or characteristics described may be combined in any suitable manner in one or more embodiments or examples.

Although embodiments of the present disclosure have been illustrated and described above, it could be understood by those skilled in the art that changes, modifications, alternatives, and variations can be made in the embodiments without departing from the scope of the present disclosure.

1. A nasopharynx stent for anti-snoring, comprising:
a nasal tube configured as a soft nasal tube; and
a balloon configured as a soft balloon, communicated with a terminal end of the nasal tube, and provided with a vent hole in communication with the nasal tube and first mesh-shaped through holes formed in a peripheral wall of the balloon.

2. The nasopharynx stent according to claim 1, wherein the nasal tube is provided with second mesh-shaped through holes.

3. The nasopharynx stent according to claim 1, further comprising a baffle connected to a front end of the nasal tube.

4. The nasopharynx stent according to claim 3, wherein the baffle is provided with a plurality of ventilation holes.

5. The nasopharynx stent according to claim 1, wherein the nasal tube comprises a first nasal tube and a second nasal tube; the balloon comprises a first balloon and a second balloon; the first balloon is in communication with a terminal end of the first nasal tube while the second balloon is in communication with a terminal end of the second nasal tube; the baffle comprises a first baffle and a second baffle; the first baffle is connected to a front end of the first nasal tube, while the second baffle is connected to a front end of the second nasal tube; the first baffle is connected with the second baffle.

6. The nasopharynx stent according to claim 1, further comprising a nose pack having an adhesive backing and connected with the baffle.

7. The nasopharynx stent according to claim 1, wherein the nasal tube and the balloon are made of medical-grade biocompatible soft materials.

8. The nasopharynx stent according to claim 3, wherein the baffle is fitted over and connected to an outer tube wall of the front end of the nasal tube, and the baffle is disposed along a radial plane of the nasal tube.

9. The nasopharynx stent according to claim 3, wherein the baffle is a ring-shaped sheet, and an inner circumference of the baffle fits with an outer wall of the nasal tube.

10. The nasopharynx stent according to claim 5, wherein the first balloon and the second balloon are both provided with vent holes and first mesh-shaped through holes.

11. The nasopharynx stent according to claim 5, wherein the first baffle is fitted over and connected to an outer tube wall of the front end of the first nasal tube, while the second baffle is fitted over and connected to an outer tube wall of the front end of the second nasal tube.

12. The nasopharynx stent according to claim 5, wherein the first baffle and the second baffle are both provided with ventilation holes.
